(19) United States
(12) Patent Application Publication
(10) Pub. No.: US 2025/0149125 A1
(43) Pub. Date: May 8, 2025

(54) METHOD AND SYSTEM FOR PREDICTING CATALYST PROPERTIES USING IMAGE ANALYSIS

(71) Applicant: UOP LLC, Des Plaines, IL (US)

(72) Inventors: Louis A. Lattanzio, Mount Prospect, IL (US); Mahesh Kumar Gellaboina, Kurnool AP (IN); Michael J. McCall, Geneva, IL (US); Howard J. Greenberg, Woodridge, IL (US); Leslie A. Esguerra, Crest Hill, IL (US)

(21) Appl. No.: 18/807,569

(22) Filed: Aug. 16, 2024

Related U.S. Application Data
(60) Provisional application No. 63/596,022, filed on Nov. 3, 2023.

(51) Int. Cl. G16C 20/30

(52) U.S. Cl. CPC G16C 20/30

(57) ABSTRACT

A system comprises an image sensor having a field of view, a catalyst sample holder disposable in the field of view of the image sensor, an image collection platform that receives image data representing a captured image, and an image analysis platform that analyzes the image data to determine a predicted property for the solid catalyst sample.

### RELATED APPLICATIONS

This application claims priority to U.S. Provisional Patent Application Ser. No. 63/596,022, filed on Nov. 3, 2023, the entirety of which is incorporated herein by reference.

### BACKGROUND

Numerous hydrocarbon conversion processes are used to alter the structure or properties of hydrocarbon streams. Such processes include isomerization from straight chain paraffinic and or olefinic hydrocarbons to more highly branched hydrocarbons, dehydrogenation for producing olefinic or aromatic compounds, reforming to produce aromatics and motor fuels, alkylation to produce commodity chemicals and motor fuels, transalkylation, and others.

Catalysis is the process of increasing the rate of a chemical reaction by adding a substance known as a catalyst. Catalysts generally react with one or more reactants in a reactor to form intermediate products that subsequently result in a final reaction product. Many such processes use catalysts to promote hydrocarbon conversion reactions. Many such catalysts are bifunctional, e.g., they contain a noble metal hydrogenation-dehydrogenation function coupled with an acidic support. These bifunctional catalyst functions transform hydrocarbons in processes such as refining. Typically, very high dispersion of the noble metal function is desired for maximum selectivity and yield. Agglomeration of noble metals reduces dispersion due to catalyst aging or poor regeneration conditions.

Catalysts tend to deactivate for a variety of reasons, including the deposition of carbonaceous material or coke upon the catalyst, sintering or agglomeration or poisoning of catalyst metals on the catalyst, and/or loss of catalytic metal promoters such as halogens. These catalysts are typically reactivated in a process called regeneration. Reactivation can thus include, for example, removing coke from the catalyst by burning (combustion), redispersing catalytic metals such as platinum on the catalyst, oxidizing such catalytic metals, reducing such catalytic metals, replenishing catalytic promoters such as halogens on the catalyst, and drying the catalyst.

The appearance of catalysts and adsorbents (generally, catalysts) often change with changes in catalyst properties. For example, deactivation is characterized by changes in metal dispersion, the breakup of catalysts particles (e.g., pills), changes in size distribution, and/or accumulation or pickup of process contaminants such as iron, sulfur (at a higher level), or chloride (at a higher level).

To monitor whether there is improper regeneration of catalysts, operators may visually inspect the appearance of the catalysts after sampling for periodic (e.g., daily or weekly) offline analysis, making qualitative yet subjective inferences on catalyst changes from visual observation. However, such analysis has varying degrees of success, e.g., based on the experience of the observer.

Alternatively, an operator may send a catalyst sample to a remotely located laboratory for other analysis, such as measuring noble metal dispersion, and the results are then returned to the operator. However, this is expensive and time consuming, and cannot provide rapid feedback that is useful for optimizing operations at runtime.

Therefore, there is a need for improved methods and systems for assessing catalyst properties based on the appearance of the catalysts.

### SUMMARY

An example system comprises an image sensor having a field of view. A catalyst sample holder is disposable in the field of view of the image sensor. The catalyst sample holder being configured to hold a solid catalyst sample therein. An image collection platform comprises one or more processors of the image collection platform coupled to the image sensor; and memory storing executable instructions that, when executed, cause the image collection platform to: receive, from the image sensor, image data representing a captured image, the captured image comprising an image of the solid catalyst sample; and transmit the image data.

The example system further comprises an image analysis platform. The image analysis platform comprises: one or more processors of the image analysis platform; and memory storing executable instructions that, when executed, cause the image analysis platform to: receive, from the image collection platform, the transmitted image data; analyze the image data to determine one or more grayscale metrics, color metrics, particle size metrics, particle shape metrics, or combinations thereof over the solid catalyst sample in the captured image; based on said one or more determined one or more grayscale metrics, color metrics, particle size metrics, particle shape metrics, or combinations determine a predicted property for the solid catalyst sample; and send an output to a receiving device based on the predicted property.

### BRIEF DESCRIPTION OF THE DRAWINGS

FIG. 1 shows an example system for analyzing a catalyst sample.

FIG. 2 shows a bottom surface of a catalyst sample holder.

FIG. 3 shows an example top surface of a catalyst sample holder including a card showing color standards and a scale.

FIG. 4 shows an example top surface of a catalyst sample holder disposed on a surface of a flatbed scanner providing an image sensor.

FIG. 5 shows an example operation that may be performed by an operator for acquiring an image of a catalyst sample using the catalyst sample holder and the image sensor.

FIG. 6 shows example features of an image collection platform.

FIG. 7 shows example features of an image analysis platform.

FIG. 8 shows an example catalyst analysis method that may be performed by the image collection platform and the image analysis platform.

FIG. 9 shows features of an example image analysis method that may be performed by the image analysis platform.

FIGS. 10A-10C shows example grayscale peak values from experimental image analysis methods and associated catalyst images.

FIG. 11 shows a set of example grayscale distributions and associated agglomerations.

FIG. 12 shows an example comparison between predicted Pt dispersion chemisorption index values and true Pt dispersion chemisorption index values in experiments.

FIG. 13 shows an example comparison of grayscale peak position and platinum agglomeration represented by dehydrogenation activity values (catalyst activity index) for a catalyst sample over a series of days on stream (DOS), and results of an x-ray diffraction (XRD) analysis of the catalyst for detecting platinum agglomeration.

FIG. 14 shows an example correlation between grayscale peak position and activity values.

FIG. 15 shows an example color classification method.

FIG. 16 shows an example dashboard for communicating catalyst analysis results over a timeline and recommendations.

FIG. 17 shows example components of an additional remote device for receiving results of catalyst sample analysis.

FIG. 18 shows an example online catalyst image collection system.

### DETAILED DESCRIPTION

Example methods and systems herein can exploit a correlation between changes in catalyst appearance and changes in one or more catalyst properties, e.g., in a standardized and objective manner. Even with some experience in attempting visual correlation, there has not been provided to date a standardized method for obtaining catalyst sample images and correlating catalyst properties with sample catalyst appearance. As a result, methods to date have increased the subjectivity and decreased the reliability in using catalyst appearance for process health.

Most commercial units depend on offline analysis to determine changes in catalyst properties, including frequent tests in commercial unit laboratories to ensure proper concentration of catalyst components and physical properties. Occasionally, other catalyst tests will be done at remote laboratories. For example, catalyst samples may be collected and transported (e.g., shipped) to a site remote from the catalyst operation for analysis. These remote other catalyst sample analysis methods typically require long turnaround times. This delay in these remote offline analyses prevents using offline analysis to track or rapidly respond to process changes, leading to poor catalyst properties and unoptimized operations using catalysts.

Example methods and systems herein can provide rapid assessment of catalyst properties. This can allow, among other example benefits, an adjustment in one or more process variables to restore optimized catalyst performance, slow deactivation, and/or better predict catalyst lifetime and replacement scheduling.

Example systems and methods herein can provide, among other things, a repeatable, standardized image capture method with image data transfer, and image analysis via models that predict catalyst properties from image attributes. Example methods can allow tuning of plant operations based on predicted catalyst properties. Other example benefits include faster turnaround in analysis of catalyst properties, more efficient process optimization, and improved prediction of catalyst lifetime and replacement. Models can be updated for further optimization based on continued use of catalyst imaging across processes. Improved models can provide even more robust prediction of catalyst properties for further improvement in process conditions. Example analysis can be performed remotely, which can also allow remote or centralized support for multiple processes.

In example methods, catalyst sample images represented by image data are collected, such as but not limited to continuous catalyst regeneration (CCR) regenerated catalyst images by an image collection platform including standardized atline, offline, or online image capture specifications or equipment. The image data is transmitted to an image analysis platform for predicting one or more catalyst properties. The image analysis platform, which may be onsite or remote (offsite), or a combination (e.g., if components are in multiple locations), processes received image data using processor-implemented example data extraction methods, which may include machine learning methods, to extract one or more image attributes from the received image data and predict catalyst properties based on the image attributes. Example image attributes include grayscale metrics, color metrics, catalyst particle size metrics, particle shape metrics, or any combination of these.

Example systems and methods can correlate catalyst appearance with catalyst properties such as metal agglomeration, catalyst damage, catalyst size attrition, or other visual characteristics. Example systems and methods, for instance, may detect the presence of impurities that can impact the color, size, and/or shape of the catalyst particles and lead to reduced performance. Predicted catalyst properties may be used to recommend one or more process attributes, e.g., process parameters and/or process changes, to improve or optimize a catalyst processing unit and/or the catalyst properties. Recommendations to a catalyst process may be determined and provided to an operator based on the predicted catalyst properties for improving catalyst and process performance, extending catalyst and equipment life, and/or enhancing process efficiency. Alternatively or additionally, predicted catalyst properties may be used to generate (or cause to be generated, e.g., via commands, prompts, or other inputs) one or more control signals, e.g., to provide an automatic, semi-automated, or assisted control optimization of an operation of a hydrocarbon conversion process, regeneration process, or other process. The control signals may be generated or caused to be generated remotely, onsite, or any combination thereof.

In a nonlimiting example, an image analysis can be used to predict platinum dispersion on catalysts as an alternative to standard offline methods to measure Pt dispersion for reforming catalyst samples. Such Pt dispersion methods include, as nonlimiting examples, chemisorption, activity testing, microscopy, and x-ray diffraction (XRD). Image analysis can be used to predict, for instance, platinum dispersion as an alternative to agglomeration activity testing, e.g., for regeneration catalyst samples.

As another example, detecting catalyst properties and providing recommendations to an operator (and/or control signals for automatic or semi-automatic control of a process) based on detected catalyst properties can improve chlorine injection consumption and dryer life by reducing excess chlorine injection through adjustments to the amount of chlorine injection (CI), e.g., based on the color of the catalyst. Ensuring the catalyst is properly regenerated can reduce heavies formation, increasing cycle life and production. An example image analysis method may output a suggestion based on the image data, providing guidance on whether chlorine injection should be increased or reduced. Yields can be improved by decreasing cracking by ensuring the catalyst is properly regenerated. If there is too much chlorine on the catalyst it can lead to cracking in the reactors. Reducing the amount of excess chlorine being injected can also minimize corrosion in a regenerator by reducing the amount of chlorine being injected. This in turn can extend cycle life, reducing the number of turnarounds or length of turnarounds due to equipment repair.

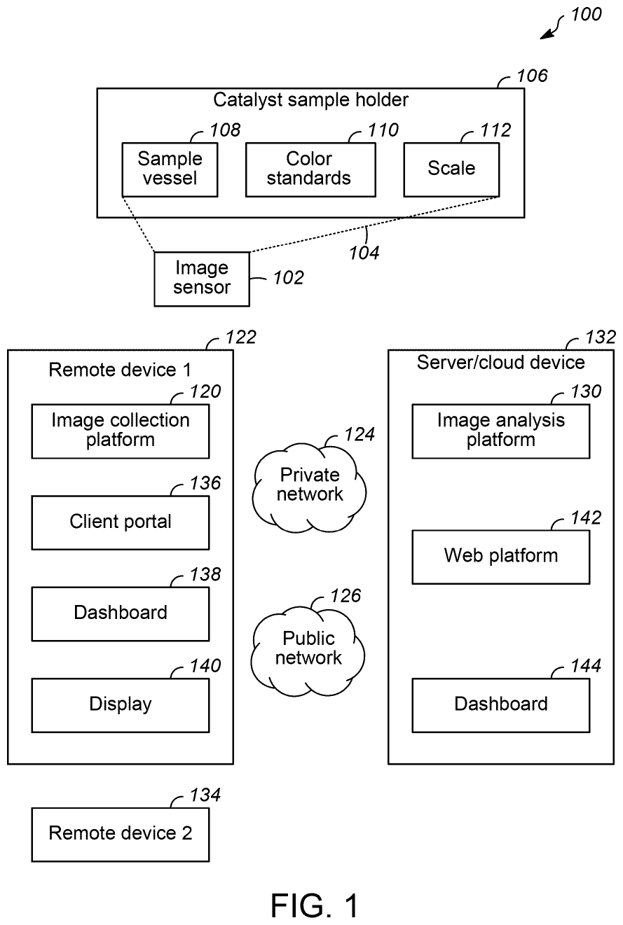

Turning now to the drawings, FIG. 1 shows an example system 100 that may be provided for analyzing a catalyst sample. A catalyst herein is intended to refer to solid catalysts and adsorbents (or a combination) that can be provided as specimens for analysis according to example methods herein, and reference to catalysts herein should be interpreted as similarly referring to adsorbents, or a combination of catalysts and adsorbents, unless directly contradicted by the description in context. Solid catalysts generally refer to catalysts that have a shape. Solid catalysts may be soft, hard, or in between to any degree.

The system 100 includes an image sensor 102 having a field of view 104. A catalyst sample holder 106 is disposable in the field of view 104 of the image sensor 102 for acquiring, e.g., capturing, one or more images of a solid catalyst sample (catalyst sample) that is provided, e.g., held or contained, within the catalyst sample holder. By disposable it is intended that the solid catalyst sample be positionable, either ex situ or in situ, within the field of view 104 of the image sensor 102. Example systems and methods for positioning the solid catalyst sample in the field of view 104 of the image sensor 102 in relatively consistent or standardized manners are provided herein.

The catalyst sample holder 106 may include, for instance, a sample vessel 108, such as but not limited to a cup, in which the catalyst sample is at least partially contained while allowing the catalyst sample to be viewable by the image sensor 102. The catalyst sample holder 106 may also be provided with or include a visible set of color standards 110 and/or a visible scale 112, which, along with the sample vessel 108, may be disposed within the field of view 104 of the image sensor 102.

The image sensor 102 is coupled via wires or wirelessly to an image collection platform 120 for collecting images produced from the image sensor. The image collection platform 120 may be provided in a remote device 122, such as a device having one or more processors and memory, a nonlimiting example being a computer. The remote device 122 may be located onsite where catalysts are in use and catalyst samples may be collected for imaging, or it may be located offsite. The image collection platform is configured to receive image data from the image sensor 102 representing a captured image that includes an image of the catalyst sample (e.g., in the sample vessel 108) and transmit the image data.

The image collection platform 120 is coupled, e.g., via a private network 124 or a public network 126, to an image analysis platform 130, which may be provided in a server or cloud device 132, such as a device having one or more processors and memory, a nonlimiting example being a computer. The image collection platform 120 and the image analysis platform 130 may be connected directly, through cloud, through a remote connection (e.g., infrastructure as a service (IaaS), software as a service (SaaS), or platform as a service (PaaS)), through a client/server arrangement, or via other connections. An example cloud connection can be part of a secure, scalable infrastructure for collecting, aggregating, and storing data, in which various components communicate, e.g., using IaaS, PaaS, data lakes, etc.

The image analysis platform 130 is configured to receive the transmitted image from the image collection platform 120, analyze the image data to determine one or more predicted properties and send an output to a receiving device based on the predicted property(s). The receiving device may be, for instance, the remote device 122 and/or a separate remote device 134. The remote device 122 (and/or the separate remote device 134) may be any processor-based device, including but not limited to a computer or a mobile device. The remote device 122 may include a client portal 136, dashboard 138, and/or display 140 for communicating (e.g., interfacing) with the server or cloud device 132. The server or cloud device 132 may include a web platform 142 and/or a dashboard 144 for communicating (e.g., interfacing) with the remote device 122 and/or remote device 134.

A nonlimiting example image sensor 102 may be provided by a charge coupled device (CCD), though any suitable image sensor may be used. The image sensor 102 may be integrated with or contained within a device such as a camera or a scanner, or it may be a standalone device. For example, the image sensor 102 and the catalyst sample holder 106 can be respectively positioned such that the sample vessel 108 (and optionally, the set of color standards 110 and/or the scale 112) can be viewed by the image sensor so as to capture substantially consistent and repeatable views (e.g., views having a substantially consistent perspective or vantage point) of catalyst samples at one or more times.

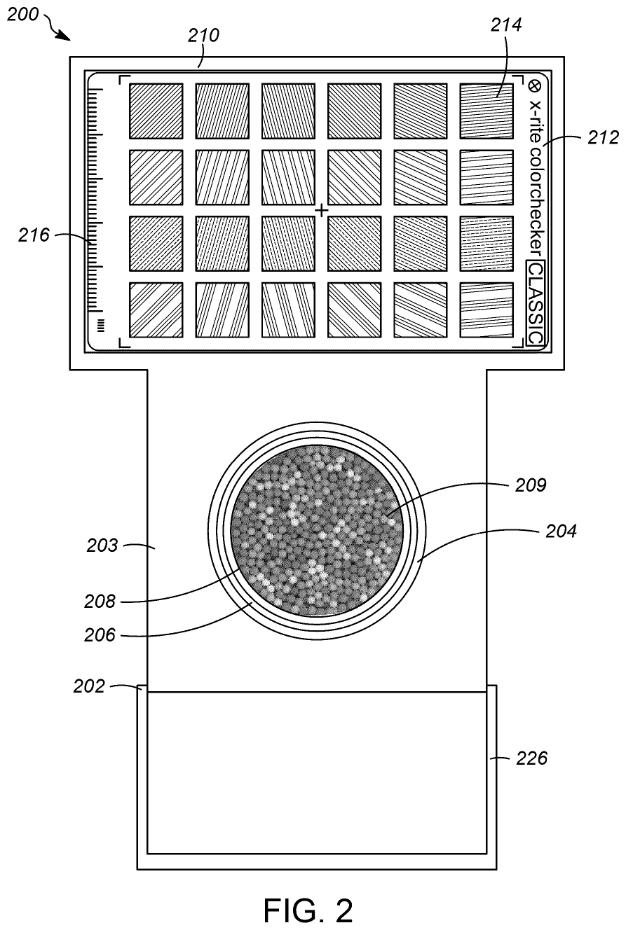

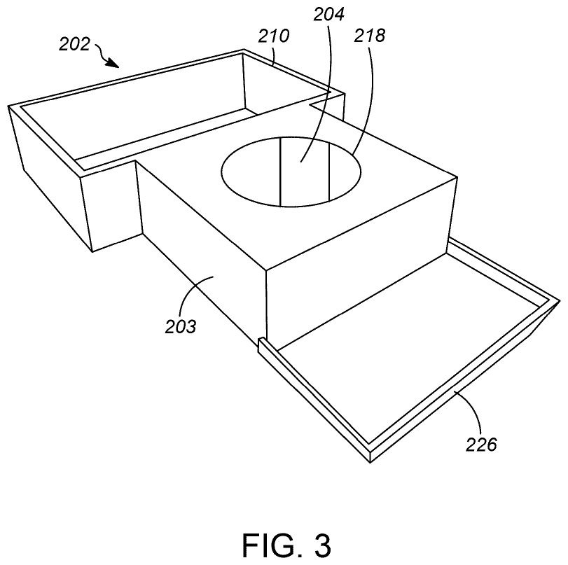

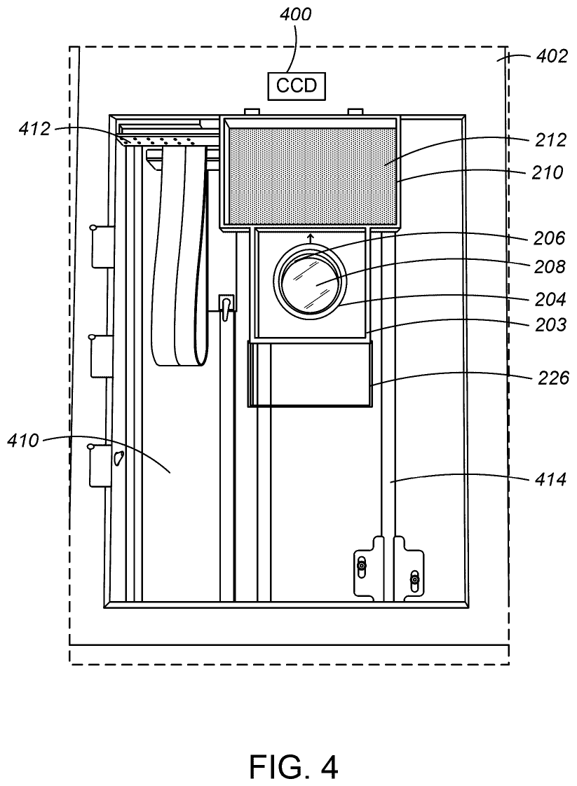

FIG. 2 shows a bottom surface of a catalyst sample holder 200, which is an example of the catalyst sample holder 106, and FIGS. 3-4 show a top surface thereof. The catalyst sample holder 200 includes a tray 202, which provides a three-dimensional template (3D template) for a sample vessel 206 and further in this example the set of color standards 110, the scale 112, and other components such as identification labels. The tray 202, which may be made of materials such as but not limited to plastic (e.g., a 3D printed or molded plastic), includes a sample vessel accommodating portion 203, which may be a generally box-shaped portion or have other three-dimensional shapes. The sample vessel accommodating portion 203 includes or surrounds an opening 204 extending therethrough, e.g., a cylindrical or frustoconical opening, for receiving at least partially therein a sample vessel 206 (an example of the sample vessel 108).

The nonlimiting example sample vessel 206 shown in FIG. 2 is embodied in an x-ray fluorescence (XRF) sample cup. The sample vessel 206 may have a transparent or semitransparent bottom end surface 208, such as a transparent film (e.g., a MYLAR film) disposed at a bottom end surface of the sample vessel, so that a catalyst sample 209 within the sample vessel is visible therethrough. The transparent or semitransparent film may be pre-assembled onto the sample vessel 206, or assembled onsite before collecting images. The opening 204 may be sized and shaped relative to the sample vessel 206, e.g., having an inner volume complementary to the outer volume of the sample vessel, so that when the sample vessel is placed within the opening, e.g., through a top end 218 as shown in FIG. 3, the sample vessel is substantially or completely seated within the opening to limit or prevent unintentional shifting of the vessel, while the bottom end surface 208 remains substantially flush with and exposed through a bottom end of the opening, as illustrated in the bottom surface of the tray 202 shown in FIG. 2.

The tray 202 may further include one or more card accommodating portions 210, e.g., including an opening defined by a peripheral surface for supporting thereon one or more cards, such as a card 212. An example color calibration card is a COLORCHECKER CLASSIC card provided by Calibrite. A bottom surface of the card 212 includes a color standards chart 214 disposed, e.g., printed thereon, which is an example of the set of color standards 110, and a scale 216 disposed, e.g., printed thereon, which is an example of the scale 112. In other embodiments, the set of color standards 110 and the scale 112 may be provided in separate cards that are placed in separate portions of the tray 202. The opening of the card accommodating portion 210 may be sized with respect to the area of the bottom-facing surface of the card 212 such that the bottom of the opening accommodates the card to limit or prevent unintentional shifting of the card while allowing the color standards chart 214 and the scale 216 to be exposed through the opening. Further, the tray 202, including the size and the location of the card accommodating portion 210 and the sample vessel accommodating portion 203, may be configured such that the bottom-facing surface of the card 212 when disposed in the card accommodating portion (e.g., inserted via a top surface as shown in FIGS. 3 and 4, or inserted via and attached to a bottom surface) is generally even with the bottom surface of the sample vessel accommodating portion 203 and the bottom surface of the sample vessel 206, as shown in FIG. 2.

The tray 202 may further include a label accommodating portion 226 for receiving a label (not shown), which may include identification or other information related to the catalyst sample or the test details. Data, such as metadata in these labels can be, but need not be, extracted and assigned to the image in the image analysis database. A nonlimiting example label is a laboratory information management system (LIMS) label. The label accommodating portion 226 may be sized and positioned such that the label is viewable by the image sensor 102.

FIG. 4 shows the tray 202 positioned relative to an image sensor (an example of image sensor 102) provided by a CCD 400 of a flatbed scanner 402, a nonlimiting example being an EPSON V600 flatbed scanner having 1200 dpi resolution and an extending cover (e.g., 1.5″ or greater). It will be appreciated that other flatbed scanners, scanners of other types, or other image sensors more generally are possible.

The sample vessel 206 is seated within the opening 204 of the sample vessel accommodating portion 203, and the card 212 is seated (face down) in the card accommodating portion 210. The transparent bottom end surface 208 of the sample vessel 206 and the card 212 are exposed to the clear surface 410 of the flatbed scanner 402. A light source 412 movable via rails 414 produces an image, which may be directed via one or more mirrors (not shown) to the CCD 400 for acquiring the image. The CCD is coupled to the image collection platform 120, e.g., via electronics (not shown) in the flatbed scanner 402 and connections such as ports (not shown) using suitable connection protocols or a wireless connection using suitable wireless protocols to upload the acquired image to the image collection platform.

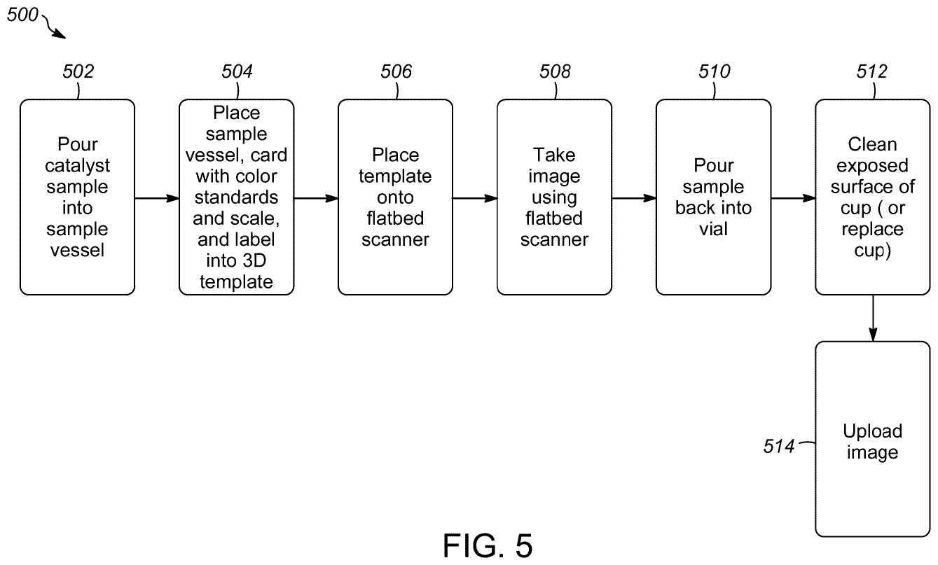

FIG. 5 shows an example operation 500 that may be performed by an operator (an operator may be provided by one or more human operators, one or more processor-based devices or systems (e.g., automated systems, computers, robots, etc.) performing example operations or steps thereof, or any combination of human and processor-based operators) for acquiring an image of a catalyst sample using the catalyst sample holder 106 and the image sensor 102. At 502, a solid catalyst sample, e.g., a multilayer sample (though a single layer sample is possible) is poured or placed, e.g., from a vial or other source, into the sample vessel 108, e.g., an XRF cup having a MYLAR film bottom as provided above, so that the catalyst sample covers the bottom end surface 208 of the sample vessel.

At 504, the sample vessel 108, and a card such as card 212 having the set of color standards 110 and the scale 112 printed thereon, are placed into a three-dimensional (3D) template such as tray 202 having the sample vessel accommodating portion 203 and the card accommodating portion 210. A LIMS label or other label for identification or other information may be placed into the label accommodating portion 226. At 506, the operator positions the 3D template into the field of view 104 of the image sensor 102 by placing the 3D template onto the clear surface 410 of the flatbed scanner 402 and operating (or initiating an operation, e.g., a conventional operation, of) the scanner. At 508, the flatbed scanner operates (e.g., in a typical scanning operation for flatbed scanners) to capture (e.g., scan) an image of at least the bottom end surface 208 of the sample vessel, the card 212, and the LIMS label. An example scan acquires an interior image of the 3D template at, say, 1200 dpi with no color correction algorithms, although the resolution can vary depending on the scanner and/or scanner configuration, and one or more correction algorithms may be used if needed.

The operator may pour the catalyst sample back into the vial at 510 and may clean the bottom end surface 208 (e.g., wipe off the MYLAR film of the XRF cup, if used) or replace the sample vessel 108. At 514, the acquired image may be uploaded (manually, automatically (e.g., in response to being recognized as an acquired image), or any combination) from the image sensor (e.g., in the flatbed scanner) to the image collection platform 120 as image data.

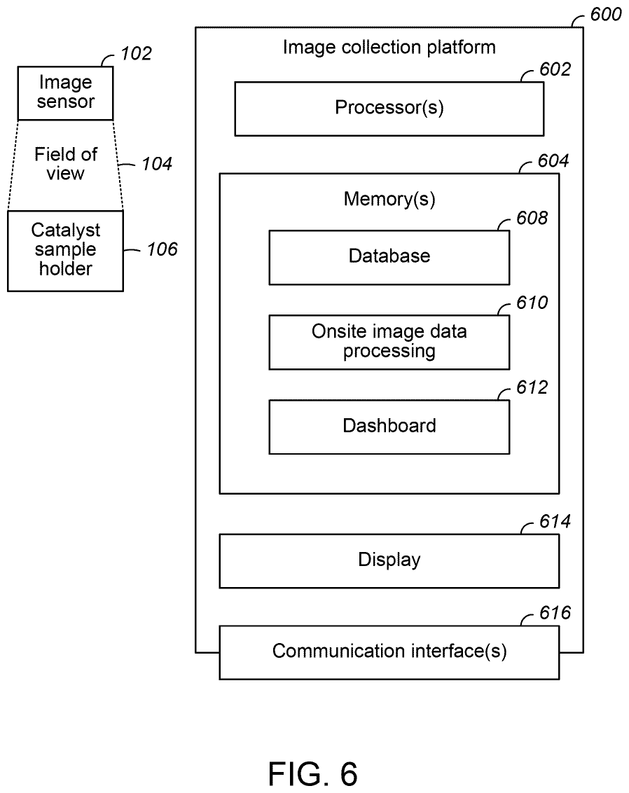

FIG. 6 shows example features of an image collection platform 600, which may correspond to image collection platform 120 and may include other features of the remote device 122. The image collection platform 600 includes one or more processors 602 and a memory 604 that includes a database 606 for data storage. The memory 604 can store executable instructions for providing an onsite image data processing module 610 and optionally a dashboard 612 that may correspond to dashboard 138. The image collection platform 120 and/or the remote device 122 may further include a display 614 corresponding to display 140 and/or a communication interface 616 for interfacing with the image sensor 102, the server/cloud device 132, and/or the additional remote device 134.

The memory 604, including the onsite image data processing module 610, stores instructions executable for causing the processor 602 of the image collection platform to receive image data from the image sensor 102 representing the captured image. The captured image includes at least an image of the solid catalyst sample in the sample vessel 108 (e.g., the catalyst sample visible through the bottom end surface 208 of sample vessel 204) and optionally also the color standards 110, the scale 112, and/or the label (not shown). The stored instructions when executed further cause the processor 602 to transmit the image data to the image analysis platform 130, e.g., to the server/cloud device 132 via the private network 124 or the public network 126. The image data may be, but need not be, processed by the image collection platform 120 before it is transmitted. Image processing tasks may be shared by the image collection platform 120 and the image analysis platform 130 in any suitable distribution. For example, image processing may take place entirely offsite (by the image analysis platform 130) or at least partially onsite (by the image collection platform 120). One or multiple sets of image data representing one or multiple images, including samples of different catalysts and/or samples of the same catalyst taken at different times, may be stored in the memory (e.g., in the database 608) and transmitted as a batch or individually.

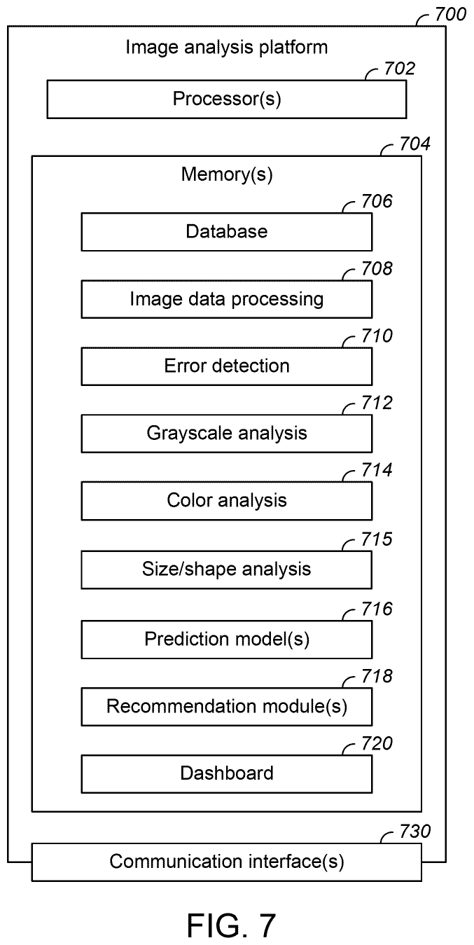

FIG. 7 shows example features of an image analysis platform 700, which may correspond to image analysis platform 130 and may include other features of the server/cloud device 132. The image analysis platform 700 includes one or more processors 702 and a memory 704 that may include a database 706 for data storage (including storage of prior analysis or received image data), and may store executable instructions for providing modules such as an image data processing module 708, an error detection module 710, a grayscale analysis module 712, a color analysis module 714, a size/shape analysis module 715, a prediction model 716, a recommendation module 718, and a dashboard 720 that may correspond to dashboard 144. The image analysis platform 700 and/or the server/cloud device 132 may also include communication interface 730 for interfacing with the remote device 122 and optionally additional remote device 134. The communication interface 730 may include the web platform 142.

The memory 704 stores instructions executable for causing the processor 702 of the image analysis platform 702 to receive, e.g., via the communication interface 730, image data transmitted from the image collection platform 120, analyze, e.g., via the grayscale analysis module 712, color analysis module 714 or size/shape analysis module 715, one or more grayscale metrics, color metrics, particle size metrics, particle shape metrics, or any combination thereof over the solid catalyst sample in the captured image, determine, e.g., using the prediction model 716, a predicted property for the solid catalyst sample, and send an output, e.g., via the communication interface 730 and optionally further via the recommendation module(s) 718 and the dashboard 720, an output to a receiving device such as the remote device 122 or the additional remote device 134 based on the predicted property.

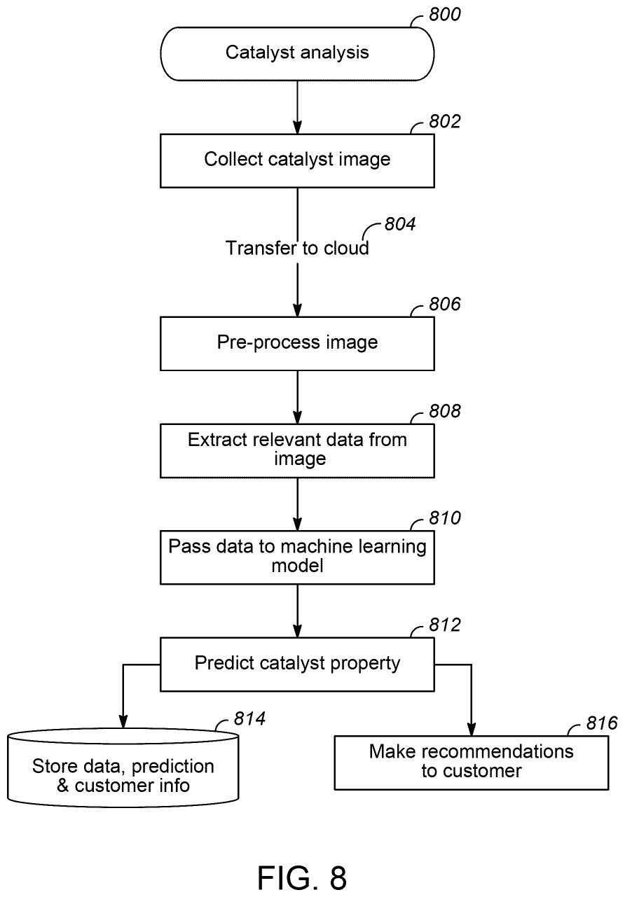

FIG. 8 shows an example catalyst analysis method 800 that may be performed by the image collection platform 600 and the image analysis platform 700. The example analysis method 800 may be performed for each of one or more solid catalyst samples acquired at one or more associated times. The image collection platform 600 collects an image of a solid catalyst sample at 802, e.g., from image sensor 102, as image data and transmits, e.g., via communication interface 616 and optionally dashboard 612, the image represented by image data at 804 to the image analysis platform 700, which in a nonlimiting example is located in the cloud. The image analysis platform 700 pre-processes the received image as needed at 806, e.g., via image data processing module 708, and extracts relevant data from the image at 808, such as but not limited to a grayscale metric via grayscale analysis module 712, a color metric via color analysis module 714, a solid catalyst particle size and/or shape metric via size/shape analysis module 715, or other data (including combinations). The image analysis platform 700 passes the extracted data to a machine learning model at 810, and the machine learning model, e.g., prediction model 716, processes the extracted data to predict one or more catalyst properties at 812. The image data, extracted data, prediction, and/or associated information such as but not limited to customer or operator information, may be stored at 814, e.g., in the database 706. The image analysis platform 700 may further generate and output one or more recommendations at 816 to a customer (operator), e.g., via the recommendation module 718, dashboard 720, and communication interface 730.

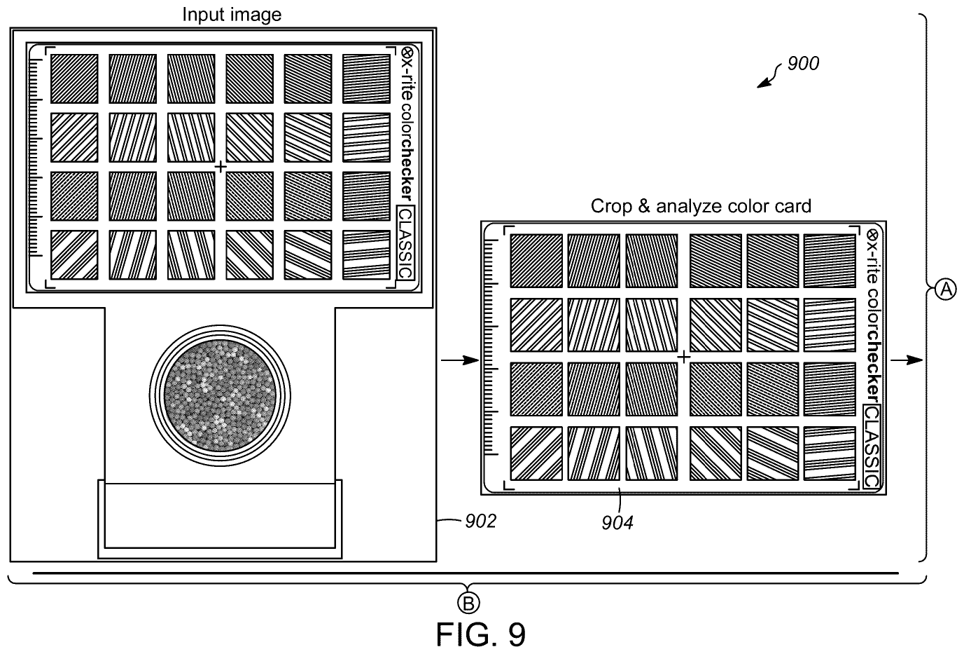

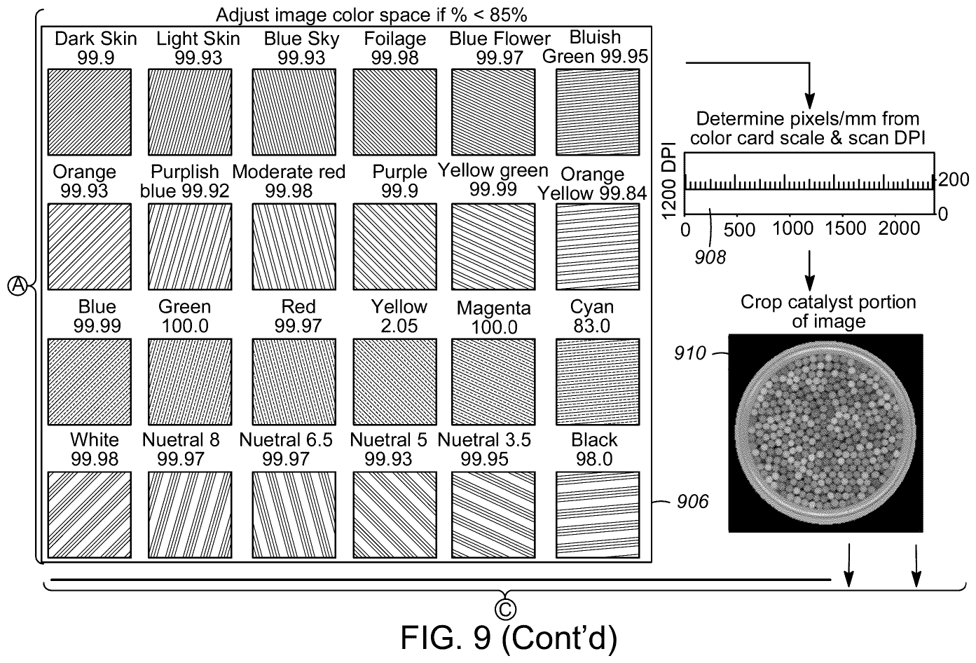

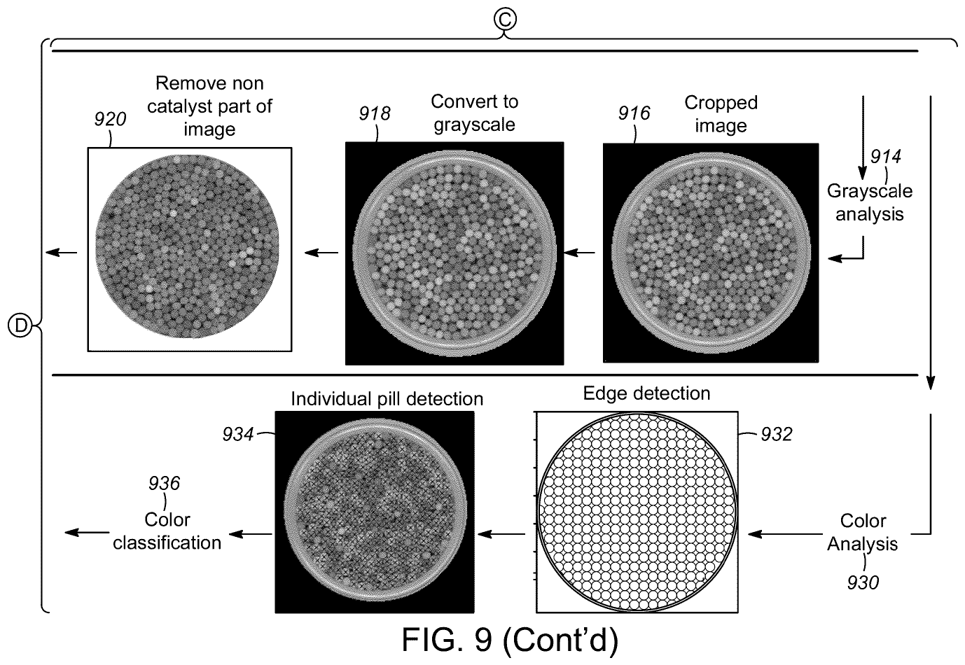

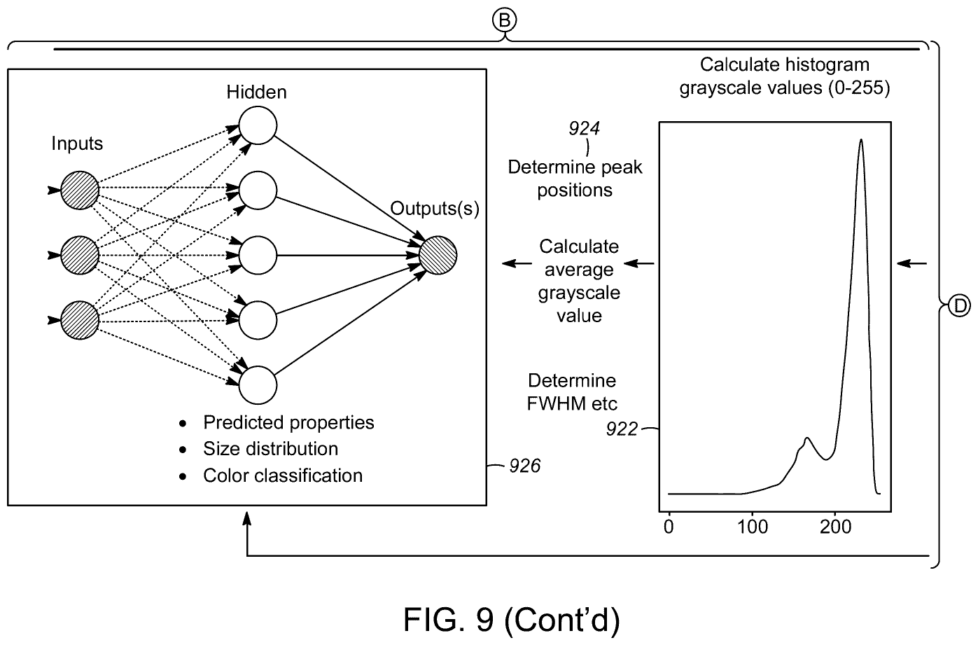

FIG. 9 shows features of an example image analysis method 900 that may be performed by the image analysis platform 700 in image preprocessing step 806, data extraction step 808, data passing step 810, and catalyst property prediction step 812. Example image analysis methods may include any or all of the features from the example image analysis method 900. In the example image preprocessing 806, input image data representing an image of the catalyst sample disposed at the bottom end surface 208 of the sample vessel 206, and of the card 212 including the color standards chart 214 and the scale 216, is received at 902. The input image may further include the label (not shown). At 904, the color standards chart 214 and the scale 216 are isolated, e.g., cropped, and analyzed, e.g., for calibration and/or for error detection, using known image feature detection and image cropping methods. If it is not possible to obtain the standards chart 214 or the scale 216 via cropping at 904, it may be determined that the image is insufficient, and an error notification (and request to submit a new image) may be transmitted to the image collection platform 120.

In an example analysis method, each color patch (e.g., square) in the color

standards chart 214 can be extracted at 904 and imaged colors (e.g., RGB) in one or more portions or subimages (including the entire color patch) are converted to values, e.g., values in a LAB color space (e.g., with values for lightness and color dimensions) or other color system. The values for each color patch may be compared to values for associated defined colors, e.g., using Euclidean distance. A correspondence between the color patch and the associated defined color can be determined, for instance, by determining the percentage of pixels (or of other portions or subimages) in the color patch matching the defined color, or by another metric. At 906, if the percentage of matching pixels (or other correspondence metric) falls below a threshold, e.g., 85% (greater or smaller thresholds are possible), the image color space can be adjusted by adjusting the image data to more closely align the image with the defined colors. If the correspondence metric falls below a lower threshold, e.g., 50%, it may be determined that the image is insufficient, and an error notification (and request to submit a new image) may be transmitted to the image collection platform 120.

At 908, the extracted scale 216 can be used along with a known resolution of the image sensor, e.g., scanner dpi, to determine a pixel density in the image such as in pixels/mm. For example, for an example scale 216 having 50 mm length and for an image sensor resolution of 1200 pixels/inch, the scale may include 2367 pixels (px). A pixel density (in the example equation below, pixelsPerMetric) may be calculated as 2367 px/50 mm=47.35 px/mm. For a 1600 pixels/inch resolution, the 50 mm scale may include 3155 px, and pixelsPerMetric=3155 px/50 mm=63.1 px/mm. Similarly, for 2400 pixels/inch resolution, the 50 mm scale may include 4734 pixels, and pixelsPerMetric=94.7 px/mm.

At 910, the portion of the extracted image including the catalyst sample is isolated, e.g., cropped, from the image using known edge detection and cropping methods. The catalyst sample portion of the image, for example, may be defined by an identified (inner or outer) edge of the imaged bottom end of the sample vessel 206. The example cropped image shown in FIG. 9, for instance, encompasses the outer circular surface (e.g., edge or rim) at the bottom end of the sample vessel 206 to include the entire bottom end surface of the sample vessel.

The cropped catalyst sample image obtained from step 910 is then analyzed to extract one or more features. For example, FIG. 9 shows a grayscale analysis method 914 for determining a grayscale metric by analyzing the cropped catalyst sample image 916. At 918, the cropped catalyst sample image 916 is converted to grayscale (e.g., using known image processing methods), and at 920, the non-catalyst part of the cropped image, e.g., the edge or rim of the sample vessel 206 and the bottom surface of the sample vessel accommodating portion 203, are removed or cropped (for instance) to isolate the (two-dimensional) image of the catalyst sample.

At 922, a distribution of grayscale values is determined over a plurality of subimages or over a plurality of imaged catalyst particles. For instance, the isolated image of the catalyst sample from step 920 can be divided either into subimages, e.g., defined by individual pixels or by groups of pixels (squares, rectangles, or any other shape). For each pixel or each group of pixels, a grayscale value is determined, e.g., from 0-255, and a distribution of the grayscale values is determined over the pixels or groups.

As another example, the individual catalyst particles in the isolated image of the catalyst sample from step 920 are identified, e.g., using edge or feature detection methods. Grayscale values are determined for individual catalyst particles, where for each individual catalyst particle, or one or more portions thereof, one or more grayscale values may be determined (e.g., for one or more portions of each particle, including pixels or groups of pixels within each particle) and combined. A distribution of the grayscale values over the imaged catalyst particles or portions thereof is then determined. Particle size metrics, e.g., sizes of the identified individual catalyst particles or size distributions, can also be determined, e.g., using the determined pixel density. Similarly, particle shape metrics can be determined, e.g., from the identified catalyst particles, the detected edges or features, the determined pixel density, etc. alone or in combination with known shape determination methods (e.g., geometric analytic methods, predictive or classification methods, image analysis, shape matching, etc.).

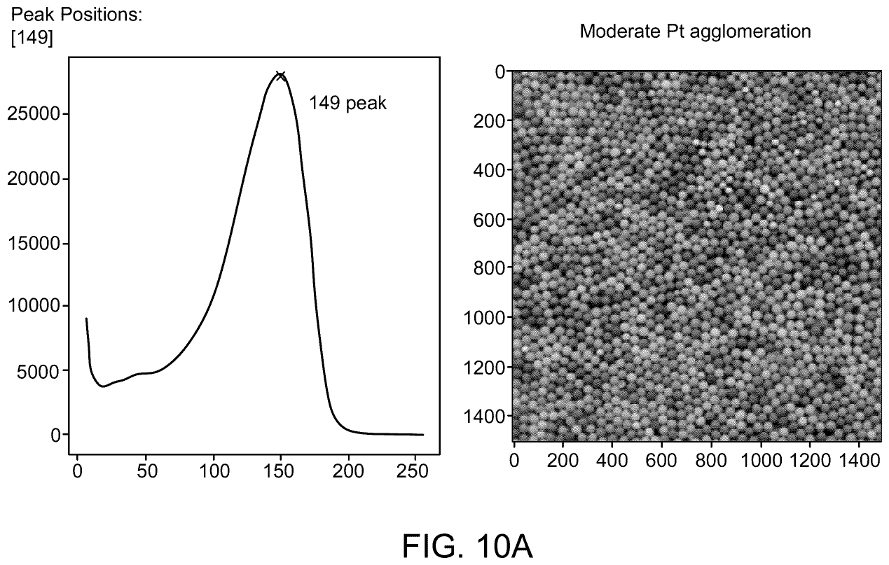

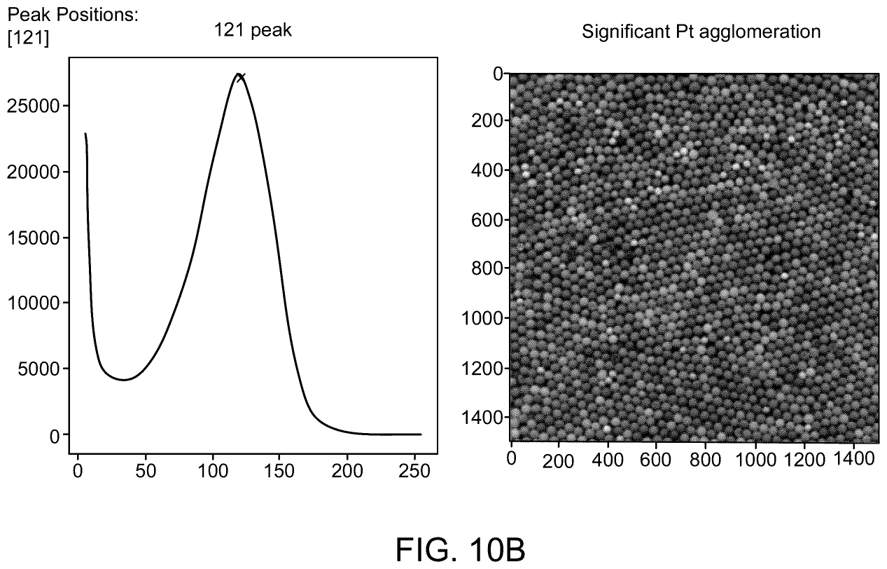

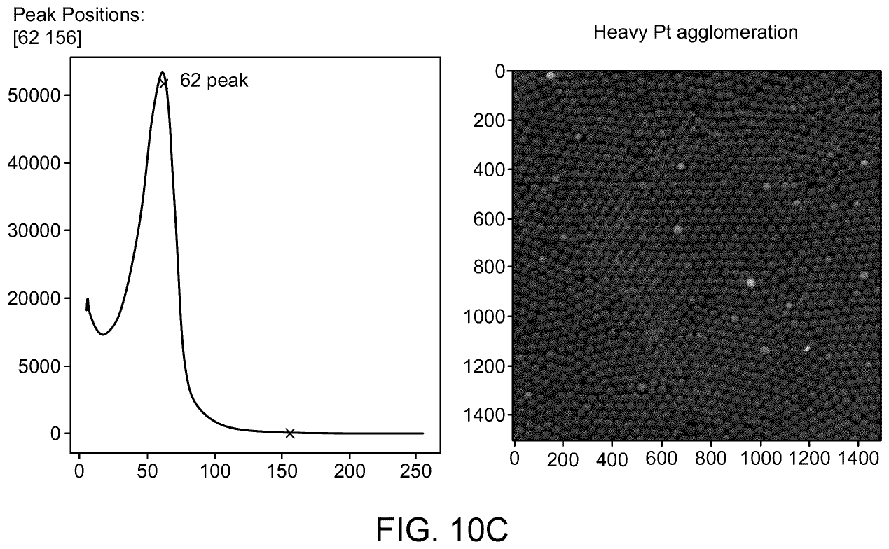

Determining the distribution of grayscale values may include generating a histogram of grayscale values across pixels, groups of pixels, or individual catalyst particles. For example, FIGS. 10A-10C show an example histogram of grayscale values, e.g., using a standard (0-255) scale, across pixels for each of three catalyst samples taken at three different and progressively later days of service (DOS), along with a portion of the cropped grayscale image of the catalyst sample.

The distribution of grayscale values determined in step 922 may then be used to determine one or more grayscale metrics. For example, at step 924, one or more of peak positions, average grayscale value, full width at half maximum (FWHM), or other metric can be determined by processing the distribution of grayscale values. For example, FIG. 10A shows a grayscale peak value of 149, FIG. 10B shows a grayscale peak value of 121, and FIG. 10C shows a grayscale peak value of 62.

At 926, one or more of the determined grayscale metrics, along with any other features that may be considered (if any) such as but not limited to determined size metrics, shape metrics, and/or determined color metrics (as described in further detail below) are input to the prediction model 716 for predicting one or more catalyst properties. The prediction model 716 may be embodied in a machine learning model, which may include a neural network including an input layer, one or more hidden layers, and an output layer, with updatable parameters (e.g., weights). The grayscale metrics, color metrics, size metrics, shape metrics, or other inputs may be input as one or more vectors in the input layer or represented in other ways. Inputs may be embedded using one or more embedding layers and/or may be encoded using one or more encoders. Nodes in the neural network layers may be selected any suitable functions. Hyperparameters for the machine learning model may be selected in any suitable manner. The output layer may provide, for instance, a predicted value (e.g., via regression) or a classification as an output.

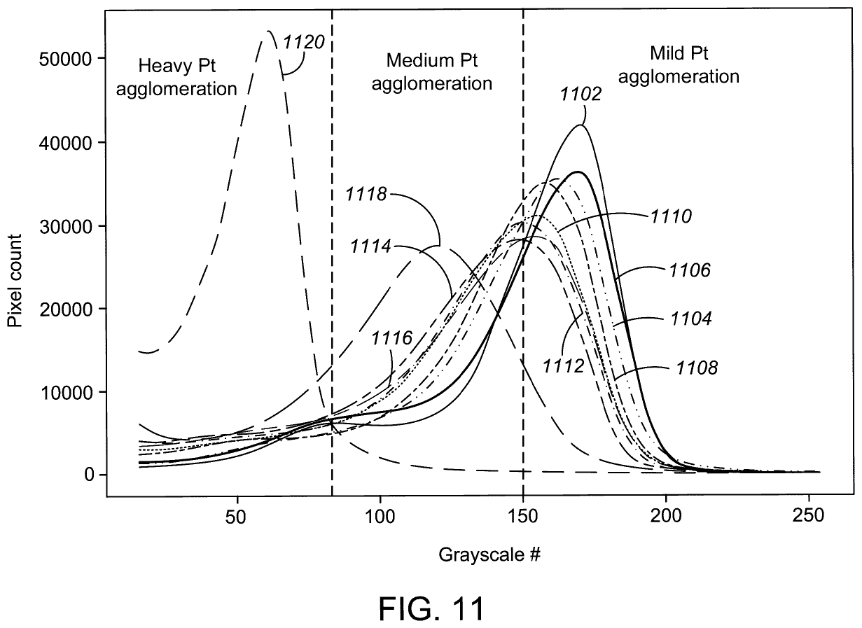

For example, a neural network model may be configured and trained to predict platinum agglomeration, as may be represented by a Pt dispersion chemisorption value, activity value, or other values, from grayscale peak values. In FIG. 10A, the grayscale peak value of 149 is input to an example neural network model, which corresponds to an actual Pt dispersion chemisorption value representing mild agglomeration. In FIGS. 10B and 10C, with the input grayscale peak values of 121 and 62, respectively, the example neural network model generates predicted Pt dispersion chemisorption values representing significant and high Pt agglomeration respectively. FIG. 11 shows a set of example grayscale distributions and associated predicted Pt dispersion chemisorption values corresponding to minimal (e.g., lines 1102, 1104, 1106, 1108, 1110, 1112), mild (e.g., lines 1118, 1114, 1116), and significant (e.g., line 1120) Pt agglomeration respectively.

In an example method for training the machine learning model, catalyst samples are received for determining a predicted property, catalyst agglomeration, such as may be represented by a Pt dispersion chemisorption value. Catalyst sample images are acquired from one or more remote sites, e.g., using a standardized image acquisition method using the catalyst sample holder 106 and a flatbed scanner as provided herein, and collected using the image collection platform 120. The images are uploaded as image data to the server/cloud device 132, e.g., to cloud storage. The received images are processed using the image analysis platform 130, e.g., by determining a grayscale distribution and one or more grayscale metrics and predicting the agglomeration, e.g., a Pt dispersion chemisorption value or catalyst activity index. An actual (ground truth) Pt dispersion chemisorption value can be determined, e.g., using methods known to those of ordinary skill in the art. The predicted Pt dispersion chemisorption value can be compared to the actual Pt dispersion chemisorption value and a loss can be determined. The losses determined over a plurality of catalyst samples can be used to update the parameters of the machine learning model using known machine learning optimization methods.

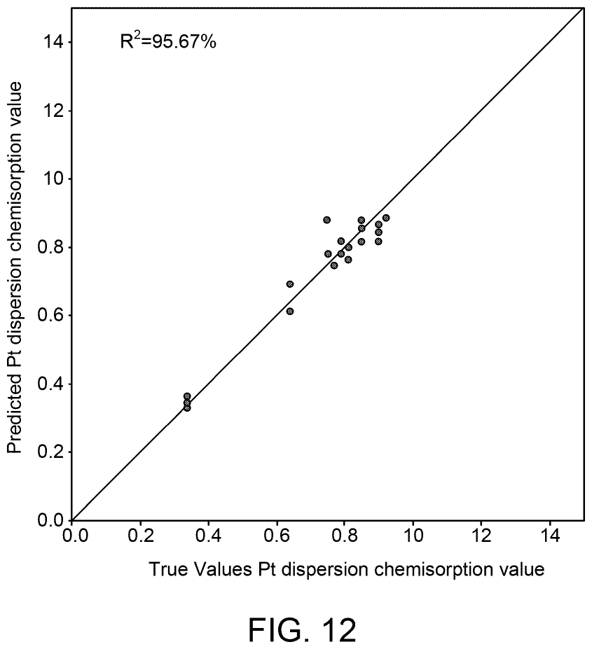

FIG. 12 shows an example comparison between predicted Pt dispersion chemisorption values and true Pt dispersion chemisorption values in experiments. The strong correlation between the Pt dispersion chemisorption values illustrates that example machine learning models can provide useful Pt dispersion chemisorption predictions.

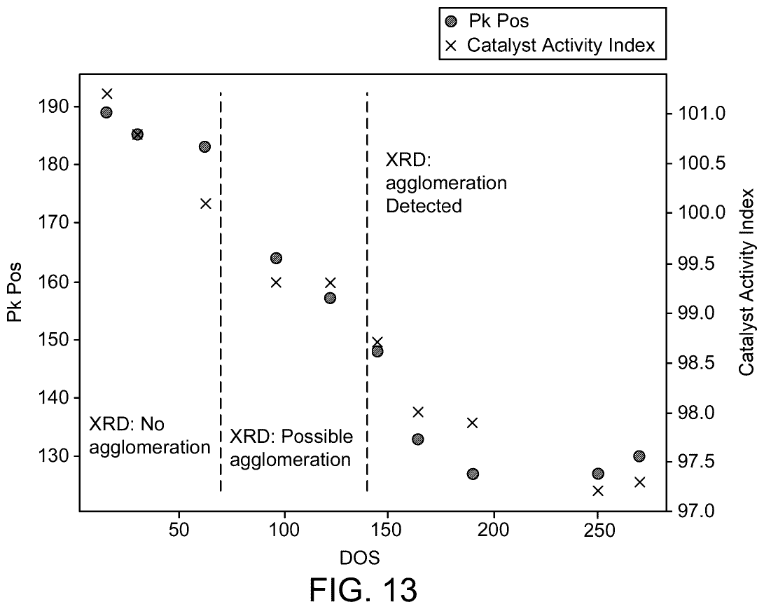

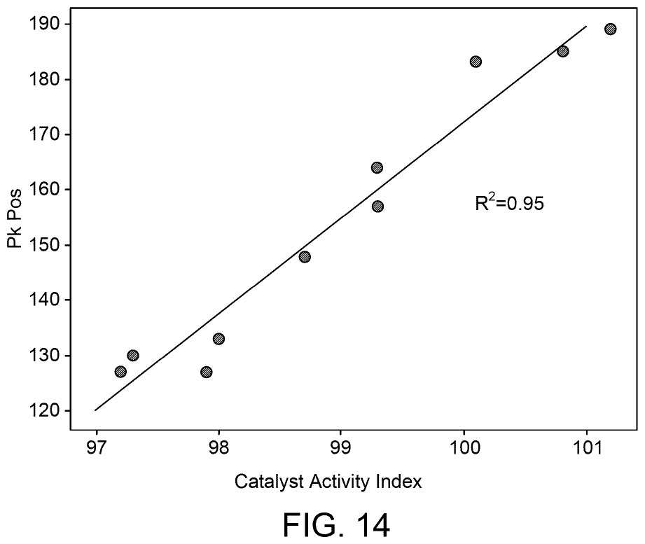

FIG. 13 shows an example comparison of grayscale peak position and platinum agglomeration represented by activity values (combi activity) for a catalyst sample over a series of days on stream (DOS), and further indicating results of an x-ray diffraction (XRD) analysis of the catalyst for detecting platinum agglomeration. As shown in FIG. 13, high grayscale peak positions (over 180) correlated to higher activity and no measured agglomeration. Lower grayscale peak positions (under 150) correlated to lower activity and detected agglomeration, while intermediate grayscale peak positions correlated to intermediate activity and possible detected agglomeration. FIG. 14 shows an example correlation between grayscale peak position and activity values, illustrating that a good correlation is present.

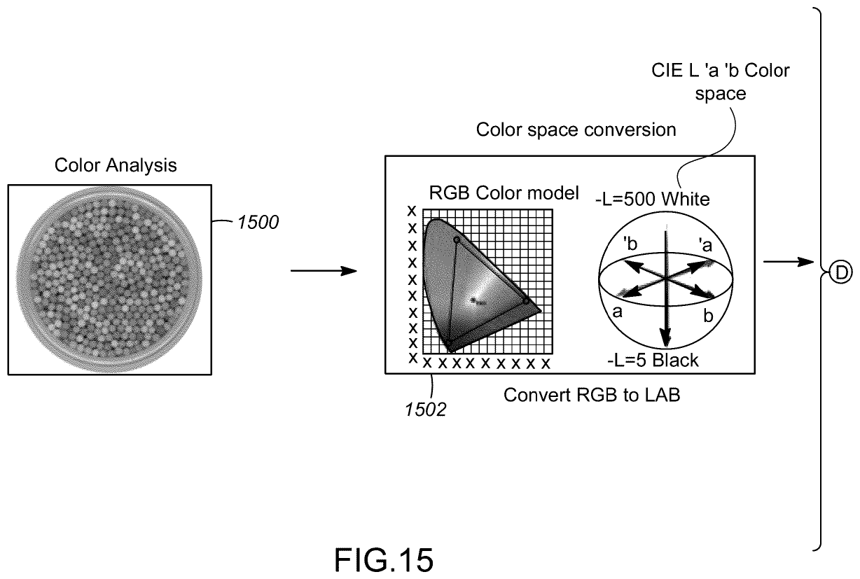

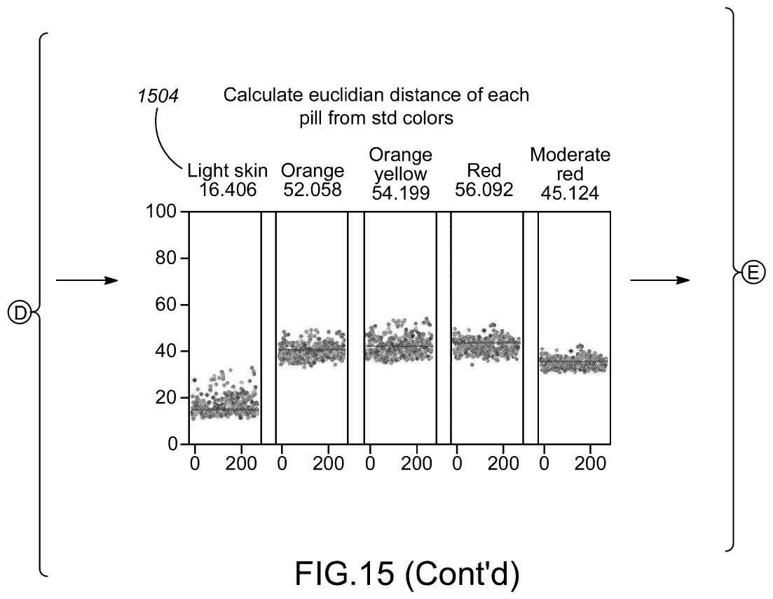

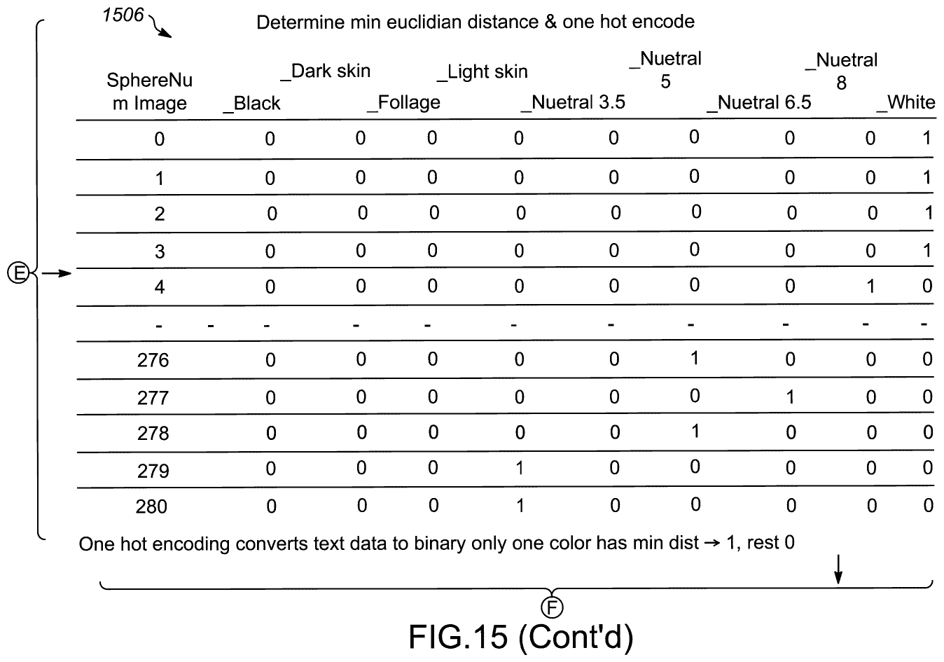

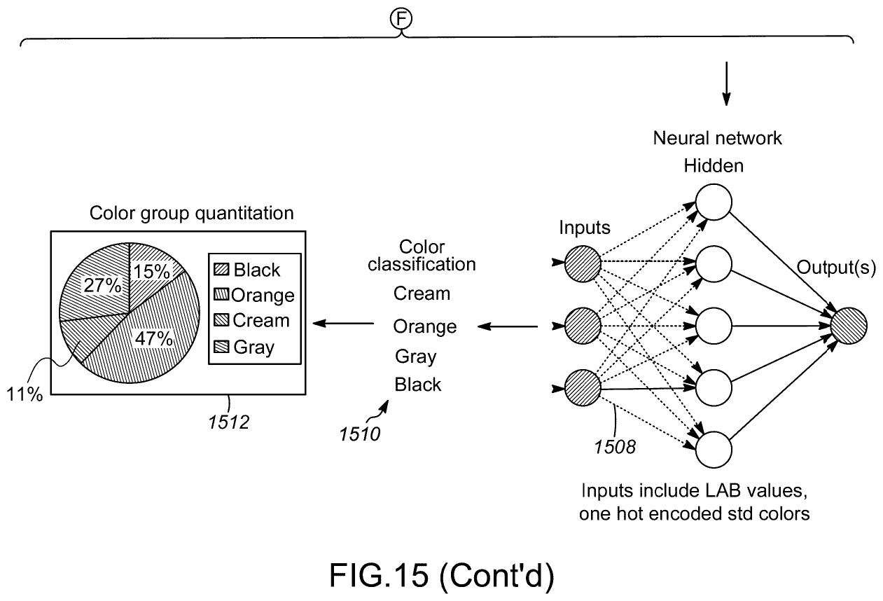

Alternatively or additionally to determining a grayscale metric(s) size metric(s), and/or shape metrics, example systems and methods can determine one or more color metrics using an example color analysis method 930 as shown in FIG. 9 and in FIG. 15. In the example color analysis method 930 the isolated catalyst portion of the image obtained at 910 is analyzed in an edge detection step 932 to identify outer edges of the sample vessel 206 and individual catalyst particles, e.g., pills. The edge detection step 932 may be performed, for example, using a Canny algorithm or other suitable image analysis algorithms. The detected edges are then used to detect individual catalyst particles in the isolated catalyst portion at 934.

The detected individual catalyst particles are then analyzed in a color classification 936, an example of which is illustrated in the color analysis method 1500 in FIG. 15. At 1502, imaged colors (e.g., RGB) for each detected catalyst particle in the isolated catalyst portion of the image are converted to values, e.g., values in a LAB color space or other color system. This may be performed, for instance using similar or different methods as disclosed herein for color calibration step 904. At 1504, the values for each color patch may be compared to values for a plurality of standard colors, e.g., by calculating a difference, e.g., a Euclidean distance, in the color space between the RGB color for each detected catalyst particle and each of the standard colors. For instance, as shown in FIG. 15, Euclidian distances are shown for each of a plurality of example detected catalyst particles from the nonlimiting example colors light skin, orange, orange yellow, red, and moderate red.

At 1506, for each detected catalyst particle a standard color having a minimum difference, e.g., Euclidean distance, is determined. The result of this determination is encoded, e.g., using a one-hot encoding in which, in a vector for each catalyst particle, the standard color having minimum Euclidean distance is set to one and all other standard colors are set to zero. This provides a binary representation for the standard colors for each detected catalyst particle.

At 1508, the (e.g., one hot) encoded standard colors and/or color space values (e.g., LAB values) are input into the prediction model, corresponding to step 926 in FIG. 9. At 1510, the prediction model outputs a prediction of a color classification or color group classification. In FIG. 15, the nonlimiting example color classification outputs are cream, orange, gray, and black. For example, at 1512, an example distribution (quantitation) of color groups is shown by percentage of detected catalyst particles. The output colors can be correlated with one or more additional catalyst properties. As nonlimiting examples, the color classifications of individual catalyst particles or their distributions can be used to recognize catalysts that have been permanently deactivated, such as heel catalysts. Alternatively or additionally, combinations of catalyst properties, such as color and size, color and shape, grayscale and size, color/shape/size, etc., can be used to identify catalysts that have been permanently deactivated through phase changes of the catalyst support, such as white dwarfs (e.g., which may shrink in size). Changes in catalyst properties can also be used to determine catalyst particle homogeneity, which can be monitored (for instance, over a time series) to help determine overall catalyst performance and/or cycle life, to alert an operator to change regeneration conditions, or for other applications. Further, by also detecting individual catalyst particles, e.g., at 934, and determining a pixel density, e.g., at 908, a size distribution of individual catalyst particles can be calculated. Shapes of one or more individual particles, shape distributions, etc. may also be determined, e.g., calculated, predicted, classified, compared, etc. using the detected individual particles, determined pixel density, detected particle edges, etc.

The predicted catalyst properties, whether by grayscale analysis, color analysis, size analysis, shape analysis, and/or other image analysis, can be used in one or more downstream operations, such as storage (e.g., at step 814) and/or making one or more recommendations (e.g., step 816). For example, if the predicted catalyst properties indicate a problem with a catalyst or operation, the image analysis platform 130 may generate a flag or alert, or control signal (or one or more outputs, commands, or prompts or causing one or more control signal to be generated), which may be stored along with identifying information for the operator and/or used to notify the operator The alert may be output via the dashboard, e.g., via dashboard module 144 and/or may output as a direct alert (e.g., an auto exception alert) to the operator via the remote device 122, 134, or via an alternate communication path. Detected alert conditions may be verified by other methods before an alert is generated, or an alert may be automatically generated and additional information (explanation, recommendation, etc.) provided.

As another example, the predicted catalyst properties may be used to provide an estimate of useful catalyst cycle life. For instance, the concentration of contaminants from a catalyst process or an amount of irreversibly deactivated catalysts may be tracked, e.g., in a time series, to predict or help predict an end of useful catalyst life. This can be used to help an operator determine when to replace a catalyst load and/or to remove a portion of the catalyst load and replace the portion with fresh catalyst, which in turn can boost operational performance and extend catalyst cycle life.

The image analysis platform 130, e.g., recommendation module(s) 718, may recommend further analysis (either image analysis or other catalyst analysis) and/or recommend one or more process changes based on the predicted catalyst properties. As a nonlimiting example a database (e.g., database 706) of process issues may be accessed and one or more process issues associated with the predicted catalyst properties identified. Identification may be based on one analyzed catalyst sample or over a temporal series of catalyst samples. Prior catalyst sample results may be accessed and compared to newer results for generating a recommendation. The stored process issues can be used to provide further improvement of the catalyst process and/or the catalyst. Generating recommendations may be performed using machine learning models, decision models (e.g., decision trees), database searches, and/or any other suitable method or algorithm. A separate review of the catalyst sample image, the catalyst sample, and/or other catalyst process parameters by an operator, e.g., located at a location onsite or offsite from the catalyst operation, may alternatively or additionally be used to supplement the results or recommendations.

As another example downstream operation, the predicted catalyst properties may be compared to catalyst analysis performed via other means (e.g., XRD analysis) and/or other image analysis. Stored images, e.g., in an image library, can also be used for comparisons to predicted properties or for other downstream analysis. Comparisons may be used to further improve, train, or optimize the prediction models.

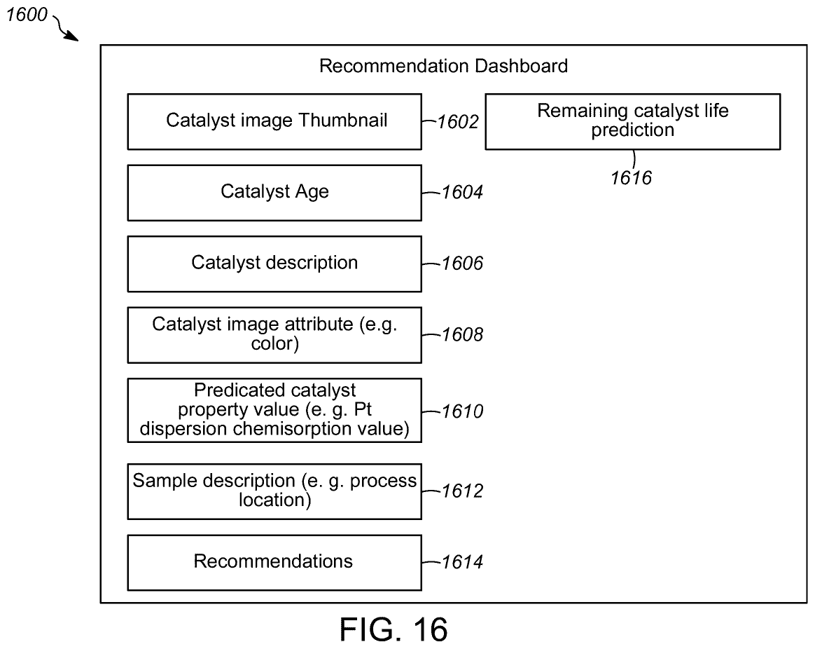

FIG. 16 shows a dashboard 1600, which may be generated from the dashboard module 144 that may be provided by the image analysis platform 132 for displaying results of catalyst image analysis and/or recommendations. The dashboard 1600 may be output, for instance, over private network 124 or public network 126 to the remote device 122 having dashboard 138. The dashboard may be displayed on a display 140 of the remote device 122.

The example dashboard 1600 may include any of various displayed items, e.g., via windows, frames, widgets, icons, or any other suitable display component, relating to the predicted output(s), recommendations, operation controls, etc. Multiples of example items of the same or similar type or items presenting similar information may be displayed, for instance, to provide a snapshot or a timeline of catalyst life over days on stream (DOS), one or more processes, one or more control parameters, and/or one or more recommendations in one or various formats. Data represented by the timeline or any portion thereof may be stored and retrieved as needed for display, further analysis, or downstream operations.

The example dashboard 1600 may include a catalyst image thumbnail 1602, e.g., showing the imaged catalyst sample or portion thereof, a catalyst age 1604, a catalyst description 1606, a catalyst image attribute (e.g., color, shape, etc.) 1608, one or more predicted catalyst property values (e.g., Pt dispersion chemisorption value) 1610, a catalyst sample description (e.g., process location) 1612, recommendation(s) 1614, and remaining catalyst life prediction 1616. Another example item that may be displayed include, but are not limited to, a life management window showing a stored timeline of catalyst life (e.g., over days on stream (DOS)), which timeline may but need not include other displayed data such as a predicted mass percentage. Still another example item that may be displayed includes a sample results window for displaying a stored temporal series of catalyst samples (e.g., used to generate a timeline), annotations (provided from any source), associated days on stream, number of regeneration cycles, catalyst type, predicted agglomeration (supplemented using other analysis if available), short description of the sample, and others. The catalyst sample analysis results may be associated with operators in storage for retrieval, analysis, and/or for automated operation controls. Results over a plurality of operators may also be used for aggregate analysis, e.g., to improve processes or example prediction models.

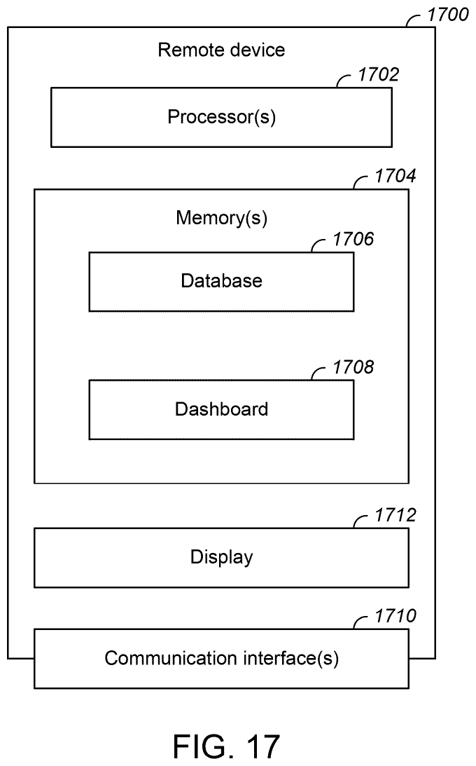

Alternatively or additionally, the dashboard may be output to and displayed on the additional remote device 134. FIG. 17 shows example components of a remote device 1700 such as but not limited to a mobile communication device, which may correspond to the additional remote device 134. The remote device 1700 includes a processor 1702 and a memory 1704. The memory 1704 includes a database 1706 and machine-executable instructions for providing a dashboard 1708 that may interface with the dashboard module 144 of the image analysis platform 132 via a communication interface 1710. The additional remote device 1700 may also include a display 1712 for displaying results and interfacing with the image analysis platform 132.

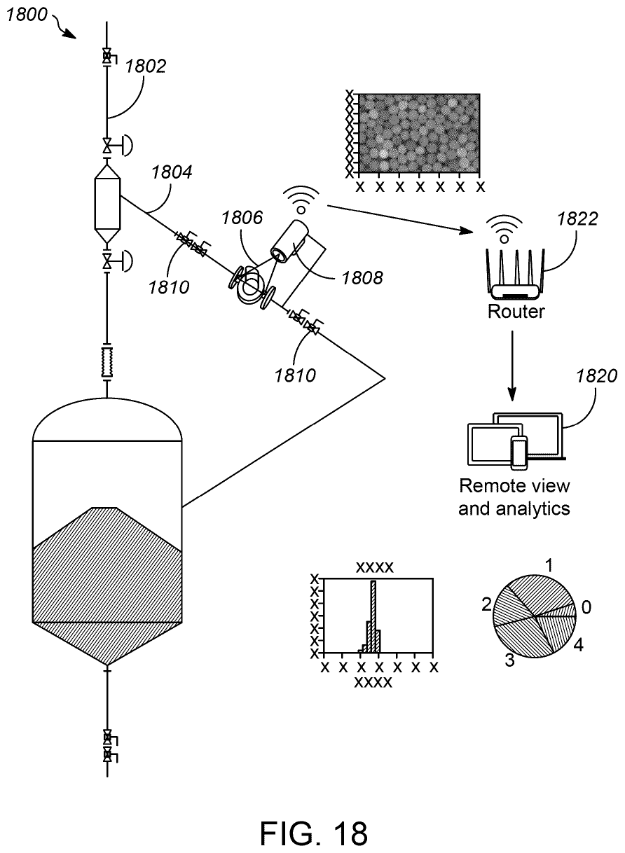

Although some example systems allow for offline collection of images of samples, it is also possible to acquire images of catalyst samples in other ways, inline or online. FIG. 18 shows an example online catalyst image collection system 1800 for acquiring an image of a catalyst sample.

In the example collection system 1800, a slip stream of catalyst 1802 is diverted through a line 1804 having a sight glass 1806. The sight glass 1806 is operatively connected to an image sensor such as a camera 1808 with suitable light provided so that the image sensor can perceive images of the catalyst. A nitrogen stream may be provided to flow counter current to flow of the catalyst to remove dust from the collection system 1800. A valving system including one or more valves 1810 is provided to allow the catalyst to flow and to hold the catalyst during image acquisition, and then opened to return catalyst to the process, so that images of the catalyst may be acquired while reducing motion blur. In an example operation, the image sensor 1808 is activated, e.g., triggered, when a valve signal is active. Image data representing the acquired images can then be transmitted to the image analysis platform 1820 either directly, e.g., via a direct wireless or wired connection, or indirectly, e.g., via a wireless transmitter such as a wireless router 1822. Valves may then be used to return the catalyst to the process. Alternatively or additionally to online image capture, a sight glass may be provided in one or more existing process lines, valves, etc., for accommodating or permitting inline image capture.

While the following is described in conjunction with specific embodiments, it will be understood that this description is intended to illustrate and not limit the scope of the preceding description and the appended claims.

Any of the lines, conduits, units, devices, vessels, surrounding environments, zones or similar provided herein may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

The computing device of a system unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by the controller or a computing device.

The methods and steps described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems for control gas flow to a burner described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

### SPECIFIC EMBODIMENTS

While the following is described in conjunction with specific embodiments, it will be understood that this description is intended to illustrate and not limit the scope of the preceding description and the appended claims.

A first embodiment of the invention is a system comprising an image sensor having a field of view; a catalyst sample holder disposable in the field of view of the image sensor, the catalyst sample holder being configured to hold a solid catalyst sample therein; an image collection platform, comprising one or more processors of the image collection platform coupled to the image sensor; memory storing executable instructions that, when executed, cause the image collection platform to receive, from the image sensor, image data representing a captured image, the captured image comprising an image of the solid catalyst sample; and transmit the image data; and an image analysis platform, comprising one or more processors of the image analysis platform; and memory storing executable instructions that, when executed, cause the image analysis platform to receive, from the image collection platform, the transmitted image data; analyze the image data to determine one or more grayscale metrics, color metrics, particle size metrics, particle shape metrics, or combinations thereof over the solid catalyst sample in the captured image; based on the determined one or more grayscale metrics, color metrics, particle size metrics, particle shape metrics, or combinations, determine a predicted property for the solid catalyst sample; and send an output to a receiving device based on the predicted property. An embodiment of the invention is one, any or all of prior embodiments in this paragraph up through the first embodiment in this paragraph, wherein the analyzing the image data comprises determining, from the image data, a distribution of grayscale values over a plurality of subimages or over a plurality of imaged catalyst particles in the solid catalyst sample; and determining the grayscale metric from the distribution of grayscale values; wherein the grayscale metric comprises a peak grayscale value, an average grayscale value, or a full width at half-maximum (FWHM). An embodiment of the invention is one, any or all of prior embodiments in this paragraph up through the first embodiment in this paragraph, wherein the analyzing the image data comprises determining, from the image data, a distribution of color values over a plurality of subimages or over a plurality of imaged catalyst particles in the solid catalyst sample; and determining the color metric from the distribution of color values; wherein the color metric comprises a Euclidean distance of respective color values in the distribution of color values from predetermined color values. An embodiment of the invention is one, any or all of prior embodiments in this paragraph up through the first embodiment in this paragraph, wherein the determining a predicted property comprises processing the determined one or more grayscale metrics, color metrics, particle size metrics, particle shape metrics, or combinations using a machine learning model. An embodiment of the invention is one, any or all of prior embodiments in this paragraph up through the first embodiment in this paragraph, wherein the machine learning model comprises a neural network. An embodiment of the invention is one, any or all of prior embodiments in this paragraph up through the first embodiment in this paragraph, wherein the analyzing the image data further comprises isolating the image of the solid catalyst sample from the captured image. An embodiment of the invention is one, any or all of prior embodiments in this paragraph up through the first embodiment in this paragraph, wherein the analyzing the image data comprises determining the size metric from the image data, wherein the size metric comprises sizes of individual catalyst particles in the plurality of imaged catalyst particles in the solid catalyst sample. An embodiment of the invention is one, any or all of prior embodiments in this paragraph up through the first embodiment in this paragraph, wherein the captured image further comprises an image of a plurality of color standards. An embodiment of the invention is one, any or all of prior embodiments in this paragraph up through the first embodiment in this paragraph, wherein the analyzing the image data comprises determining, from the image data, a distribution of color values over a plurality of subimages or over a plurality of imaged catalyst particles in the solid catalyst sample; and determining the color metric from the distribution of color values; wherein the color metric comprises a Euclidean distance of respective color values in the distribution of color values from predetermined color values; and wherein the predetermined color values are determined by extracting color standards from the image of the plurality of color standards; and converting the extracted color standards to the predetermined color values. An embodiment of the invention is one, any or all of prior embodiments in this paragraph up through the first embodiment in this paragraph, wherein the analyzing the image data further comprises extracting color standards from the image of the plurality of color standards; comparing the extracted color standards to a set of defined colors to determine a similarity; and adjusting an image color space of the captured image if the determined similarity falls under a threshold. An embodiment of the invention is one, any or all of prior embodiments in this paragraph up through the first embodiment in this paragraph, wherein the analyzing the image data further comprises detecting an error in the captured image; and transmitting a notification of the detected error to the receiving device. An embodiment of the invention is one, any or all of prior embodiments in this paragraph up through the first embodiment in this paragraph, wherein the image of the plurality of color standards is coupled to the catalyst sample holder. An embodiment of the invention is one, any or all of prior embodiments in this paragraph up through the first embodiment in this paragraph, wherein the catalyst sample holder comprises a vessel having a transparent or semitransparent surface. An embodiment of the invention is one, any or all of prior embodiments in this paragraph up through the first embodiment in this paragraph, wherein the catalyst sample holder further comprises a tray having an opening for receiving the vessel while leaving the solid catalyst sample exposed to the field of view of the image sensor. An embodiment of the invention is one, any or all of prior embodiments in this paragraph up through the first embodiment in this paragraph, wherein the analyzing the image data further comprises determining a pixel density in the captured image based on a resolution of the image sensor. An embodiment of the invention is one, any or all of prior embodiments in this paragraph up through the first embodiment in this paragraph, wherein the captured image further comprises an image of a scale; and wherein the analyzing the image data further comprises determining a pixel density in the captured image based on the image of the scale and a resolution of the image sensor. An embodiment of the invention is one, any or all of prior embodiments in this paragraph up through the first embodiment in this paragraph, wherein the output comprises one or more of a predicted state of the solid catalyst sample; and a recommended action. An embodiment of the invention is one, any or all of prior embodiments in this paragraph up through the first embodiment in this paragraph, wherein the image collection platform and the image analysis platform are connected via a network. An embodiment of the invention is one, any or all of prior embodiments in this paragraph up through the first embodiment in this paragraph, wherein the image collection platform, the image analysis platform, and the receiving device are connected via a network; and wherein the receiving device comprises a display; one or more processors of the receiving device; and memory storing executable instructions that, when executed, cause the receiving device to display on the display the sent output.

A second embodiment of the invention is a method for analyzing a catalyst sample, the method comprising receiving, by an image analysis platform, image data for a captured image obtained by an image sensor comprising an image of a catalyst sample; analyzing the received image data to determine one or more grayscale metrics, color metrics, particle size metrics, particle shape metrics, or combinations thereof over the catalyst sample in the captured image; based on the determined one or more grayscale metrics, color metrics, particle size metrics, particle shape metrics, or combinations determining a predicted property for the catalyst sample; and sending an output to a receiving device based on the predicted property. An embodiment of the invention is one, any or all of prior embodiments in this paragraph up through the second embodiment in this paragraph, wherein the analyzing the image data comprises determining, from the image data, a distribution of grayscale values over a plurality of subimages or over a plurality of imaged catalyst particles in the catalyst sample; and determining the grayscale metric from the distribution of grayscale values; wherein the grayscale metric comprises a peak grayscale value, an average grayscale value, or a full width at half-maximum (FWHM). An embodiment of the invention is one, any or all of prior embodiments in this paragraph up through the second embodiment in this paragraph, wherein the analyzing the image data comprises determining, from the image data, a distribution of color values over a plurality of subimages or over a plurality of imaged catalyst particles in the catalyst sample; and determining the color metric from the distribution of color values; wherein the color metric comprises a Euclidean distance of respective color values in the distribution of color values from predetermined color values. An embodiment of the invention is one, any or all of prior embodiments in this paragraph up through the second embodiment in this paragraph, wherein the analyzing the image data comprises determining, from the image data, sizes of individual catalyst particles in the plurality of imaged catalyst particles in the catalyst sample. An embodiment of the invention is one, any or all of prior embodiments in this paragraph up through the second embodiment in this paragraph, wherein the method further comprises receiving, by the image analysis platform, image data for one or more additional captured images obtained by the image sensor over a plurality of time steps, each of the one or more additional captured images comprising an image of a different catalyst sample; determining a plurality of grayscale metrics, color metrics, size metrics, shape metrics, or combinations thereof, wherein the determining comprises, for each additional captured image, analyzing the received image data to determine one or more grayscale metrics, color metrics, particle size metrics, particle shape metrics, or combinations thereof over the different catalyst sample in the captured image; based on the determined plurality of grayscale metrics, color metrics, size metrics, shape metrics, or combinations determining a predicted property for the catalyst sample; and sending an output to a receiving device based on the predicted property. An embodiment of the invention is one, any or all of prior embodiments in this paragraph up through the second embodiment in this paragraph, wherein the determining a predicted property for the catalyst sample comprises comparing the determined plurality of grayscale metrics, color metrics, size metrics, and/or shape metrics and determining a predicted property change. An embodiment of the invention is one, any or all of prior embodiments in this paragraph up through the second embodiment in this paragraph, wherein the determining a predicted property change comprises correlating the determining plurality of grayscale metrics, color metrics, size metrics, and/or shape metrics with at least one additional process variable. An embodiment of the invention is one, any or all of prior embodiments in this paragraph up through the second embodiment in this paragraph, wherein the output comprises feedback on an impact of changes in the at least one additional process variable on the catalyst sample. An embodiment of the invention is one, any or all of prior embodiments in this paragraph up through the second embodiment in this paragraph, wherein the output comprises an alert.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A system comprising:
an image sensor having a field of view;
a catalyst sample holder disposable in the field of view of the image sensor, the catalyst sample holder being configured to hold a solid catalyst sample therein;
an image collection platform, comprising:
one or more processors of the image collection platform coupled to said image sensor;
memory storing executable instructions that, when executed, cause the image collection platform to:
receive, from the image sensor, image data representing a captured image, the captured image comprising an image of the solid catalyst sample; and
transmit the image data; and
an image analysis platform, comprising:
one or more processors of the image analysis platform; and
memory storing executable instructions that, when executed, cause the image analysis platform to:
receive, from the image collection platform, the transmitted image data;
analyze the image data to determine one or more grayscale metrics, color metrics, particle size metrics, particle shape metrics, or combinations thereof over the solid catalyst sample in the captured image;
based on said determined one or more grayscale metrics, color metrics, particle size metrics, particle shape metrics, or combinations, determine a predicted property for the solid catalyst sample; and
send an output to a receiving device based on the predicted property.

2. The system of claim 1, wherein said analyzing the image data comprises:
determining, from the image data, a distribution of grayscale values over a plurality of subimages or over a plurality of imaged catalyst particles in the solid catalyst sample; and
determining the grayscale metric from the distribution of grayscale values;
wherein the grayscale metric comprises a peak grayscale value, an average grayscale value, or a full width at half-maximum (FWHM).

3. The system of claim 1, wherein said analyzing the image data comprises:
determining, from the image data, a distribution of color values over a plurality of subimages or over a plurality of imaged catalyst particles in the solid catalyst sample; and
determining the color metric from the distribution of color values;
wherein the color metric comprises a Euclidean distance of respective color values in the distribution of color values from predetermined color values.

4. The system of claim 1, wherein said determining a predicted property comprises processing the determined one or more grayscale metrics, color metrics, particle size metrics, particle shape metrics, or combinations using a machine learning model.

5. The system of claim 1, wherein said analyzing the image data further comprises isolating the image of the solid catalyst sample from the captured image.

6. The system of claim 1, wherein said analyzing the image data comprises determining the size metric from the image data, wherein the size metric comprises sizes of individual catalyst particles in the plurality of imaged catalyst particles in the solid catalyst sample.

7. The system of claim 1, wherein the captured image further comprises an image of a plurality of color standards;
wherein said analyzing the image data comprises:
determining, from the image data, a distribution of color values over a plurality of subimages or over a plurality of imaged catalyst particles in the solid catalyst sample; and
determining the color metric from the distribution of color values;
wherein the color metric comprises a Euclidean distance of respective color values in the distribution of color values from predetermined color values; and
wherein the predetermined color values are determined by:
extracting color standards from the image of the plurality of color standards; and
converting the extracted color standards to the predetermined color values;
wherein said analyzing the image data further comprises:
extracting color standards from the image of the plurality of color standards;
comparing the extracted color standards to a set of defined colors to determine a similarity; and
adjusting an image color space of the captured image if the determined similarity falls under a threshold.

8. The system of claim 1, wherein said analyzing the image data further comprises:
detecting an error in the captured image; and
transmitting a notification of the detected error to the receiving device.

9. The system of claim 1, wherein the catalyst sample holder comprises a vessel having a transparent or semitransparent surface;
wherein the catalyst sample holder further comprises a tray having an opening for receiving the vessel while leaving the solid catalyst sample exposed to the field of view of the image sensor.

10. The system of claim 1, wherein said analyzing the image data further comprises:
determining a pixel density in the captured image based on a resolution of the image sensor;
wherein the captured image further comprises an image of a scale; and
wherein said analyzing the image data further comprises:
determining a pixel density in the captured image based on the image of the scale and a resolution of the image sensor.

11. The system of claim 1, wherein the output comprises one or more of:
a predicted state of the solid catalyst sample; and
a recommended action.

12. The system of claim 1, wherein said image collection platform and said image analysis platform are connected via a network.

13. The system of claim 1, wherein said image collection platform, said image analysis platform, and said receiving device are connected via a network; and wherein said receiving device comprises:
a display;
one or more processors of the receiving device; and
memory storing executable instructions that, when executed, cause the receiving device to:
display on the display the sent output.

14. A method for analyzing a catalyst sample, the method comprising:
receiving, by an image analysis platform, image data for a captured image obtained by an image sensor comprising an image of a catalyst sample;
analyzing the received image data to determine one or more grayscale metrics, color metrics, particle size metrics, particle shape metrics, or combinations thereof over the catalyst sample in the captured image;
based on said determined one or more grayscale metrics, color metrics, particle size metrics, particle shape metrics, or combinations, determining a predicted property for the catalyst sample; and
sending an output to a receiving device based on the predicted property.

15. The method of claim 14, wherein said analyzing the image data comprises:
determining, from the image data, a distribution of grayscale values over a plurality of subimages or over a plurality of imaged catalyst particles in the catalyst sample; and
determining the grayscale metric from the distribution of grayscale values;
wherein the grayscale metric comprises a peak grayscale value, an average grayscale value, or a full width at half-maximum (FWHM).

16. The method of claim 14, wherein said analyzing the image data comprises
determining, from the image data, a distribution of color values over a plurality of subimages or over a plurality of imaged catalyst particles in the catalyst sample; and
determining the color metric from the distribution of color values;
wherein the color metric comprises a Euclidean distance of respective color values in the distribution of color values from predetermined color values.

17. The method of claim 14, wherein said analyzing the image data comprises
determining, from the image data, sizes of individual catalyst particles in the plurality of imaged catalyst particles in the catalyst sample.

18. The method of claim 14, wherein the method further comprises:
receiving, by the image analysis platform, image data for one or more additional captured images obtained by the image sensor over a plurality of time steps, each of the one or more additional captured images comprising an image of a different catalyst sample;
determining a plurality of grayscale metrics, color metrics, size metrics, shape metrics, or combinations thereof wherein said determining comprises, for each additional captured image, analyzing the received image data to determine one or more grayscale metrics, color metrics, particle size metrics, particle shape metrics, or combinations thereof over the different catalyst sample in the captured image;
based on said determined plurality of grayscale metrics, color metrics, size metrics, shape metrics, or combinations determining a predicted property for the catalyst sample; and
sending an output to a receiving device based on the predicted property.

19. The method of claim 18, wherein said determining a predicted property for the catalyst sample comprises comparing said determined plurality of one or more grayscale metrics, color metrics, size metrics, shape metrics, or combinations and determining a predicted property change;
wherein said determining a predicted property change comprises correlating the determining plurality of grayscale metrics, color metrics, size metrics, shape metrics, or combinations with at least one additional process variable.

20. The method of claim 18, wherein the output comprises:
feedback on an impact of changes in the at least one additional process variable on the catalyst sample; and/or
an alert.
